SEQUENCE LISTING (15 sequences)

SEQ ID NO: 1 - protein, 139 aa, Homo sapiens
MASLFRSYLPAIWLLLSQLLRESLAAELRGCGPRFGKHLLSYCPMPEKTFTTTPGGWLLESGRPKEMVSTSNNKDGQALGTTSEFIPNLSPELKKPLSEGQPSLKKIILSRKKRSGRHRFDPFCCEVICDDGTSVKLCT

SEQ ID NO: 2 - protein, 22 aa, Homo sapiens
MASLFRSYLPAIWLLLSQLLRE

SEQ ID NO: 3 - protein, 30 aa, Homo sapiens
SLAAELRGCGPRFGKHLLSYCPMPEKTFTT

SEQ ID NO: 4 - protein, 50 aa, Homo sapiens
LESGRPKEMVSTSNNKDGQALGTTSEFIPNLSPELKKPLSEGQPSLKKII

SEQ ID NO: 5 - protein, 25 aa, Homo sapiens
SGRHRFDPFCCEVICDDGTSVKLCT

SEQ ID NO: 6 - DNA, 617 bp, Homo sapiens
agtctggagcccagaagggacacaccagcacagtctggtaggctacagcagcaagtctctaaagaaaggctgagaacacccagaacaggagagttcaggtccaggatggccagcctgttccggtcctatctgccagcaatctggctgctgctgagccaactccttagagaaagcctagcagcagagctgaggggatgtggtccccgatttggaaaacacttgctgtcatattgccccatgcctgagaagacattcaccaccaccccaggagggtggctgctggaatctggacgtcccaaagaaatggtgtcaacctccaacaacaaagatggacaagccttaggtacgacatcagaattcattcctaatttgtcaccagagctgaagaaaccactgtctgaagggcagccatcattgaagaaaataatactttcccgcaaaaagagaagtggacgtcacagatttgatccattctgttgtgaagtaatttgtgacgatggaacttcagttaaattatgtacatagtagagtaatcatggactggacatctcatccattctcatatgtattctcaatgacaaattcactgatgcccaattaaatgattgctgtttaaa

SEQ ID NO: 7 - DNA, 417 bp, Homo sapiens
atggccagcctgttccggtcctatctgccagcaatctggctgctgctgagccaactccttagagaaagcctagcagcagagctgaggggatgtggtccccgatttggaaaacacttgctgtcatattgccccatgcctgagaagacattcaccaccaccccaggagggtggctgctggaatctggacgtcccaaagaaatggtgtcaacctccaacaacaaagatggacaagccttaggtacgacatcagaattcattcctaatttgtcaccagagctgaagaaaccactgtctgaagggcagccatcattgaagaaaataatactttcccgcaaaaagagaagtggacgtcacagatttgatccattctgttgtgaagtaatttgtgacgatggaacttcagttaaattatgtaca

SEQ ID NO: 8 - DNA, 66 bp, Homo sapiens
atggccagcctgttccggtcctatctgccagcaatctggctgctgctgagccaactccttagagaa

SEQ ID NO: 9 - DNA, 90 bp, Homo sapiens
agcctagcagcagagctgaggggatgtggtccccgatttggaaaacacttgctgtcatattgccccatgcctgagaagacattcaccacc

SEQ ID NO: 10 - DNA, 150 bp, Homo sapiens
ctggaatctggacgtcccaaagaaatggtgtcaacctccaacaacaaagatggacaagccttaggtacgacatcagaattcattcctaatttgtcaccagagctgaagaaaccactgtctgaagggcagccatcattgaagaaaataata

SEQ ID NO: 11 - DNA, 75 bp, Homo sapiens
agtggacgtcacagatttgatccattctgttgtgaagtaatttgtgacgatggaacttcagttaaattatgtaca

SEQ ID NO: 12 - DNA, 880 bp, homo sapiens
  misc_feature: Chorionic gonadotropin, beta polypeptide 5 (CGB5)
tccagcactttgctcgggtcacggcctcctcctggctcccaggaccccaccataggcagaggcaggccttcctacaccctactccctgtgcctccaggctcgactagtccctagcactcgacgactgagtctctgagatcacttcaccgtggtctccgcctcacccttggcgctggaccagtgagaggagagggctggggcgctccgctgagccactcctgcgcccccctggccttgtctacctcttgccccccgaagggttagtgtcgagctcaccccagcatcctacaacctcctggtggccttgccgcccccacaaccccgaggtataaagccaggtacacgaggcaggggacgcaccaaggatggagatgttccaggggctgctgctgttgctgctgctgagcatgggcgggacatgggcatccaaggagccgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggactcctcttcctcaaaggcccctccccccagccttccaagtccatcccgactcccggggccctcggacaccccgatcctcccacaataaaggcttctcaatccgca

SEQ ID NO: 13 - protein, 165 aa, homo sapiens
  misc_feature: Chorionic gonadotropin, beta polypeptide 5 (CGB5)
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 14 - protein, 7 aa, Artificial
  Synthetic 7-mer peptides corresponding to residues 1 through 7 of HCGbeta encoded by type II genes beta3, beta5 and beta8
SKEPLRP

SEQ ID NO: 15 - protein, 7 aa, artificial
  Synthetic 7-mer peptides corresponding to residues 1 through 7 of HCGbeta encoded by type I gene beta7
SREMLRP

(19) United States
(12) Patent Application Publication
(10) Pub. No.: US 2015/0105281 A1
(43) Pub. Date: Apr. 16, 2015

(54) Method for the Selection of Serum Biomarkers of Epigenetic Alterations, Particularly of Global Hypomethylation and Their Uses

(71) Applicants: Universite Paris Descartes, Paris (FR); Centre National de la Recherche Scientifique (CNRS), Paris (FR); Institut Curie, Paris (FR); Institut Gustave Roussy, Villejuif (FR)

(72) Inventors: Dominique Bellet, Boulogne-Billancourt (FR); Alain Pecking, Colombes (FR); Sophie Richon, L'Isle Adam (FR)

(73) Assignees: Universite Paris Descartes, Paris (FR); Centre National de la Recherche Scientifique, Paris (FR); Institut Curie, Paris (FR); Institut Gustave Roussy, Villejuif (FR)

(21) Appl. No.: 14/394,799

(22) PCT Filed: Apr. 17, 2013
(86) PCT No.: PCT/EP2013/058015

§ 371 Date: Oct. 16, 2014

(30) Foreign Application Priority Data
Apr. 17, 2012 (EP) 12305449.6

(51) Int. Cl. C12Q 1/68; G01N 33/76; G01N 33/74; G01N 33/574

(52) U.S. Cl. CPC C12Q 1/6886; C12Q 1/6883; G01N 33/57423; G01N 33/57407; G01N 33/57446; G01N 33/76; G01N 33/74; G01N 2333/62; C12Q 2600/154; C12Q 2600/106; G01N 2800/368
USPC 506/9; 435/6.11; 436/501; 435/7.92

(57) ABSTRACT

The present invention provides a novel method for the selection of serum biomarkers of epigenetic alterations, particularly of global hypomethylation, and the use of said biomarkers in a method for screening, diagnosing and following a pathology associated to epigenetic alterations of cell in an individual, such as placental-related pathology or cancer. The present invention also relates to a method of detecting a predisposition to placental-related pathology or cancer based on the presence or the level of said biomarker in a serum or plasma sample of said patient. The present invention also relates to a method for predicting and following the effect of drugs targeting epigenetic modifications and in particular the effect of demethylating agents. The present invention is directed to a kit comprising such serum biomarkers of epigenetic alterations, particularly of global hypomethylation.

The present invention provides a novel method for the selection of serum biomarkers of epigenetic alterations, particularly of global hypomethylation, and the use of said biomarkers in a method for screening, diagnosing and following a pathology associated to epigenetic alterations of cell in an individual, such as placental-related pathology or cancer. The present invention also relates to a method of detecting a predisposition to placental-related pathology or cancer based on the presence or the level of said biomarker in a serum or plasma sample of said patient. The present invention also relates to a method for predicting the effect of drugs targeting epigenetic modifications, and in particular the effect of demethylating agents, and to a method for following the effect of drugs targeting epigenetic modifications, and in particular the effect of demethylating agents. The present invention is directed to a kit comprising such serum biomarkers of epigenetic alterations, particularly of global hypomethylation.

Although the cells in the human body contain the same DNA sequence, the function and phenotype differ [1, 2]. This implies that, apart from genetic programming, the phenotype is regulated by another phenomenon. This is called epi- (greek for upon, above)-genetic. Epigenetic inheritance is defined as cellular information, other than the DNA sequence itself, that is heritable during cell divisions. Epigenetic templates that control gene expression are transmitted to daughter cells independently of the DNA sequence. There are three main, inter-related types of epigenetic inheritance: DNA methylation, genomic imprinting and histone modification [3]. It has become increasingly apparent that epigenetic inheritance is important in many physiological and pathophysiological conditions. Epigenetic codes are much more subject to environmental influences than the DNA sequence. This could explain how lifestyle and toxic chemicals affect susceptibility to diseases. Up to 70% of the contribution to a particular disease can be nongenetic [4].

Epigenetic templates can change and be altered during normal ageing and contribute to common cancer risk in adults. The single leading risk factor for cancer is age. Although that has often been attributed to the accumulation of mutations over time, an alternative and complementary interpretation is that age itself disrupts the epigenetic programme, increasing cancer risk. Epigenetic alterations can also support clonal evolution in human cancers, contributing to tumour progression. Thus, epigenetics play key role in cancer development and come to rival genetics.

Amongst epigenetic alterations in cancer, DNA methylation alterations are divided by the contrary findings of increased methylation (hypermethylation) at specific gene promoters within a genomic environment of a general loss of methylation (hypomethylation) compared to normal cells. Cancer-associated DNA hypomethylation is as prevalent as cancer-linked hypermethylation, but these two types of epigenetic abnormalities usually seem to affect different DNA sequences. DNA hypomethylation in cancer often affects more of the genome than does hypermethylation so that net losses of genomic 5-methylcytosine (m⁵C) are seen in many human cancers [5].

It is noteworthy that extensive cancer-associated DNA hypomethylation in the human genome preceded that of cancer-linked DNA hypermethylation. However, until recently, there has been much emphasis on the critical role of DNA hypermethylation in human carcinogenesis. In comparison, there has been inadequate attention paid to decreased methylation of DNA in cancer and hypomethylation in cancer is only now going a renaissance [6]. Genome wide hypomethylation is a decrease (compared to total cytosines) from approximately 4% in most normal tissues to 2-3% in cancer tissues [7]. This change in cancer tissues was first observed in lung and colon carcinomas compared to adjacent normal tissues and in various malignancies compared to various postnatal tissues, demonstrating that overall genomic methylcytosine levels were lower in cancer tissues [8, 9]. Moreover, high-throughput genomic methylation analysis of tumours, including cancers of the stomach, kidney, colon, pancreas, liver, uterus, lung and cervix show that the frequency of hypomethylated sites is quite high [3]. Finally, high percentage of malignant tumors, especially metastases, have DNA with unusually low methylcytosine contents relative to the normal tissues.

The high frequency of cancer associated DNA hypomethylation, the nature of the affected sequences, and the absence of associations with DNA hypermethylation are consistent with an independent role for undermethylation of certain DNA sequences in cancer formation or tumor progression. In addition, hypomethylation is a mechanism of drug, toxin and viral effects in cancer and an exciting development in cancer hypomethylation involves a link to diet.

Genomic hypermethylation in cancer has been observed most often in CpG islands in gene regions. In contrast, very frequent hypomethylation is seen in both highly and moderately repeated DNA sequences in cancer. The magnitude of the decrease in methylation observed in cancers compared to matched controls (4% down to 2-3% total methyl-cytosine) is most commonly accounted for by loss of methylation at repetitive sequences [10].

Amongst highly repetitive DNA sequences that display tumor-associated hypomethylation are endogenous retrotransposons [6]. Retrotransposons, also called transposons via RNA intermediates, are genetic elements that can amplify themselves in a genome. Retrotransposons consist of two sub-types, the long terminal repeat (LTR) and the non-LTR retrotransposons. Around 42% of the human genome is made up of retrotransposons. Amongst those retrotransposons that display tumor-associated hypomethylation are Long Interspersed Nuclear Element 1 (LINE-1) retrotransposons, which are 6-kb interspersed DNA repeats. LINE-1 are non-LTR retrotransposons which makes up around 15% of the human genome.

Amongst moderately repeated DNA sequences, there is the other class of human retrotransposons that display tumor-associated hypomethylation, the LTR retrotransposons including those from endogenous retroviruses, especially, the HERV-K family [6]. While there are only about 30±50 full-length HERV-K sequences in the human genome, there are an estimated 10 000 solitary long terminal repeats (LTRs) from HERV-K. At least some of the solitary LTRs can drive reporter gene expression.

Retrotransposons or retroviral derived elements can have their transcription upregulated in vivo by DNA demethylation in both cancer and placental cells. As representative examples, high-level expression of HERV-K gag and env RNA is associated with germ cell and trophoblastic tumors. Moreover, env RNA was detected in most assayed term placental samples [7]. This latter observation may be related to the facts that much of term placental tissue is of trophoblast origin and that placental DNA displays global genomic hypomethylation. Indeed, genome wide hypomethylation decreases from approximately 4% in most normal tissues to 0.76% in normal placenta [7].

Another type of repeated DNA sequence is satellite DNAs which are tandem, high-copy-number repeats composed of variations of oligonucleotide. The hypomethylation of repeated DNA sequences may play special role in carcinogenesis such as increasing karyotype instability or affecting gene expression. Thus, for some type of cancer, DNA hypomethylation is seen as an early indicator of tumorigenesis.

In addition, single copy sequences in the genome also exhibit hypomethylation in cancer, often associated with increased expression. Several tumor- or proliferation-associated genes have been found to be hypomethylated in human cancers. Examples of genes that are affected by hypomethylation include oncogenes such as HRAS and the Cancer Testis (CT) genes which encode Cancer Testis antigens [3]. Those CT genes are expressed normally in both the testis and aberrantly in tumours. Interestingly, they are also expressed normally in the placenta [11]. Indeed, hypomethylation of various DNA sequences in cancers and placenta seems to be a common theme. As previously indicated, global genomic hypomethylation of DNA is observed in trophoblastic cells, the constituent cells of the placenta, and in tumorigenic or metastatic cancer cells [7]. This observation at the epigenomic level might be in line with the fact that placental and cancer cells share molecular circuits which are implicated in the proliferative, invasive and migratory properties of these cells [11]. Indeed, an overview of signalling circuitries used by trophoblast cells places emphasis on the many circuitries shared with those employed by cancer cells. As virtually all mammalian cells carry similar molecular machinery regulating their proliferation, differentiation and death and as most regulatory and effector components are present in a redundant form, it is not totally surprising that normal trophoblasts and malignant cells, which may have to accomplish comparable tasks to proliferate and migrate so as to ultimately invade neighbouring tissue, use, in part, similar regulatory and effector components, similar circuitries and similar mechanisms governed by similar epigenetic templates [11].

Finally, the high frequency of cancer-linked DNA hypomethylation, the nature of the affected sequences, and the absence of associations with DNA hypermethylation are consistent with an independent role for DNA undermethylation in cancer formation or tumor progression. Hypomethylation of repeated or single-copy DNA sequences has been significantly correlated with disease progression for some tumors. Furthermore, a number of studies have shown significant decreases in global DNA methylation levels with progressing tumor stage, tumor grade, or various other indicators of poor prognosis, and global DNA hypomethylation in cancer is significantly associated with hypomethylation of certain DNA repeats. In addition, for some types of cancer, DNA hypomethylation is seen as an early indicator of tumorigenesis. As a consequence in clinical practice, DNA hypomethylation analysis may be useful in detecting cancer and managing the disease [6].

Similarly, DNA hypomethylation analysis may also be useful for the screening of abnormal pregnancies. A significant factor in placental development and function is epigenetic regulation. Disturbed placental epigenetics has been demonstrated in human placental-related pathologies like small for gestational age (SGA), intrauterine growth retardation (IUGR) and pre-eclampsia [1].

Therefore, assays for DNA hypomethylation may become a clinically useful addition to hypermethylation analyses of CpG islands.

The range of methods currently available for the study of genomic methylation has expanded considerably since initial studies in the field. Traditionally, methyl-cytosine content was measured using high performance liquid chromatography (HPLC) of genomic DNA digested to individual deoxynucleosides [12]. Although this method gives an absolute quantitative measure of methylcytosine content, it is time consuming and requires a large amount of DNA. Several alternative methods have since been developed, including Southern blot [8], Methyl group acceptance assay (MAA) [13]; direct bisulphite sequencing [14]; methylation sensitive PCR (MSP) and quantitative PCR (qPCR) based method called MethyLight [15] and lastly, monoclonal antibodies against 5-methylcytosine have been developed and used to reliably stain methylated DNA in vitro.

Using these methods, epigenetic alterations have been detected in plasma and serum. These alterations appear to be valuable molecular biomarkers and have been studied at the molecular level in either circulating tumor DNA or circulating tumor cells [16]. Methods for detecting these molecular biomarkers have the key advantage of being noninvasive but are not really simple and do not fit well with a clinical utilization on a routine basis. In particular, although DNA hypomethylation is a major epigenetic alteration in cancer cells, its diagnostic use has been, up to now, limited because a reduction in DNA methylation is technically more complicated to detect than a gain of signal [17].

Currently there is a need to provide biomarkers of epigenetic alterations and/or global hypomethylation, which can be detected by non-invasive methods and which are simple to implement, such as biomarkers of epigenetic alterations which can be detected in serum or plasma at the peptide and protein biomarkers, and which can be thus used with a clinical utilization on a routine basis.

This is the object of the present invention.

The invention relates to a novel concept for the detection of epigenetic alterations in cancer and/or placental related pathologies which is to detect biomarkers of epigenetic alterations, and preferably of global hypomethylation, in serum or plasma at the peptide and protein levels. This concept of novel tests measuring serum biomarkers of epigenetic alterations, and preferably of global hypomethylation, is based on the detection in the blood of antigens expressed by both cancer and/or placental cells, and more preferably antigens expressed by both cancer and placental cells and the expression of which is associated to genome wide hypomethylation of both cancer and placental cells. These antigens belong to a novel class of antigens designed cancer/placental antigens (CP antigens). In order to be designated as CP antigen, it must be expressed in tumour and placental cells but not expressed in more than two non germ-line normal tissues. CP antigens differ from another class of antigens, the cancer/testis antigens (CT antigens). In order to be designated as CT antigen, it must be expressed in tumour cells as well as in testis and/or placenta, but not expressed in more than two non germ-line normal tissue [18]. More than 40 CT antigens have been identified. The first identified and prototypic CT antigen is MAGE-1. Its expression is also due to the genome wide demethylation of cancer cells [19]. However, CT antigens are absent in the blood stream, processed by cancer cells and presented by MHC class-1 molecules at the cell surface. CT antigens might be tumor antigens which can serve as targets for therapeutic cancer vaccines. In striking contrast with CT antigens, CP antigens are present in the blood stream and can serve as circulating biomarkers of global hypomethylation.

WO 2011/110657 describes the specific detection of hCG beta subunit type II produced by trophoblastic and neoplastic cells, the distinction between type II and type I beta subunits is useful for the diagnostic of tumors and of Down's syndrome.

WO 2009/133088 describes the use of pro-EPIL as a biomarker for the diagnostic of testicular cancer, possibly in combination with the detection of hCG beta subunit type II.

Mock et al. (2000) [34] and Mock et al. (1999) [30] describe the detection of pro-EPIL in normal and in chromosomally abnormal pregnancies, possibly in combination with the detection of hCG. They disclose an assay using two monoclonal antibodies binding the pro-EPIL peptide.

However, none of these documents describe nor suggest a method for diagnosing a pathology associated to epigenetic alteration or a pathology associated to global hypomethylation as does the present invention.

Indeed, the inventors have demonstrated that epigenetic alterations and, in particular, the global hypomethylation of both cancer and placental cells may ultimately lead to the expression of peptides and proteins which could be shed in the blood stream. These peptides and proteins may constitute surrogate biomarkers of global hypomethylation. The presence of these peptides or proteins would be a witness of the altered epigenetic templates of either cancer cells or abnormal trophoblastic cells and the sensitive and specific detection of these surrogate biomarkers of global hypomethylation might constitute novel non-invasive methods for the detection or the management of cancer and placental related diseases.

As both trophoblastic cells and tumorigenic or metastatic cancer cells display global hypomethylation, the inventors have suggested that peptides or proteins expressed by trophoblastic cells as a consequence of their global hypomethylation might also be expressed by cancer cells and be present at increased levels in the blood stream of both cancer patients and pregnant women with placental-related pathologies. This is what it has been demonstrated by the inventors.

Thus, in a first aspect, the present invention is directed to a method for selecting peptides and proteins that can serve as biomarkers of epigenetic alterations, particularly of global hypomethylation, in the serum or plasma, said method comprising the step of selecting peptides or proteins which are present in the blood stream, such as serum or plasma, as a consequence of global hypomethylation of both trophoblastic cells and/or tumorigenic or metastatic cancer cells. In a preferred embodiment, the present invention concerns a method for the selection of a serum biomarker of epigenetic alterations, particularly of global hypomethylation, said method comprising the steps of:
a) selecting a gene which is expressed (more preferably protein or peptide expression) in a cancer and/or a placental cell, preferably both in a cancer cell and in a placental cell, and whose expression in said cell is due to the hypomethylation of its expression regulation system/promoter;
b) optionally, determining the presence or the increase of the protein, or a specific fragment thereof, encoded by said gene selected in step a) in a serum or plasma sample of a patient known to exhibit a cancer or a placental-related pathology, preferably compared to a healthy patient serum or plasma sample; and
c) selecting said protein, or a specific fragment thereof, encoded by said gene as a serum biomarker of epigenetic alteration and, optionally, of global hypomethylation, whether this gene satisfy the step a) and, optionally, step b) selection.

By “a gene whose expression in said cell is due to the global hypomethylation of the cells” it is intended to designate a gene whose expression level is linked to the hypomethylation of its regulation system (or promoter) in said cell. In a particular embodiment, it is intended to designate a gene which transcription is upregulated in vivo by DNA demethylation. By “determining the presence or the increase of the protein” it is understood the determination of the presence, of the level or of the quantity of at least a compound chosen in the group consisting of: a peptide or protein biomarker, a specific fragment thereof, a nucleic acid encoding the same and a combination thereof.

By “specific fragment thereof” it is intended a fragment of the biomarker which can be specifically detected by a specific ligand of said biomarker. Non limitating examples of specific fragments are: an epitope of said biomarker specifically binding to an anti-biomarker antibody, or a fragment, analog or derivative of an antibody; a fragment of said biomarker specifically binding to a receptor for the biomarker. By “specifically binding”, “specifically binds”, “recognizing” or the like, it is intended herein that the peptide and its ligand form a complex that is relatively stable under physiological conditions. The terms “polypeptide”, “peptide” and “protein” are used interchangeably herein to refer to a polymer of amino acid residues.

By “a serum biomarker of epigenetic alterations” it is intended to designate a peptide or a protein whose level of expression is indicative of an epigenetic alteration of the genome. More precisely, it is intended to designate a peptide or a protein whose level of expression can be quantified and is different when an epigenetic alteration of the genome, such as for example the hypomethylation of at least one gene, is present.

In a preferred embodiment, the present invention concerns a method for the selection of a serum biomarker of epigenetic alterations or of a serum biomarker of global hypomethylation, said method comprising the steps of selecting a gene which is expressed in a cancer and a placental cell and whose expression in said cell is due to the hypomethylation of its expression regulation system or promoter.

In another preferred embodiment, the present invention concerns a method for the selection of a serum biomarker of epigenetic alterations or of a serum biomarker of global hypomethylation, said method comprising the steps of:
a) selecting a gene which is expressed in a cancer and a placental cell and whose expression in said cell is due to the hypomethylation of its expression regulation system or promoter; and
b) determining the presence or the increase of the protein, or a specific fragment thereof, encoded by said gene selected in step a) in a serum or plasma sample of a patient known to exhibit a cancer or a placental-related pathology.

In another preferred embodiment, the present invention concerns a method for the selection of a serum biomarker of epigenetic alterations or of a serum biomarker of global hypomethylation, said method comprising the steps of:
a) selecting a gene which is expressed in a cancer and a placental cell and whose expression in said cell is due to the hypomethylation of its expression regulation system or promoter;
b) determining the presence or the increase of the protein, or a specific fragment thereof, encoded by said gene selected in step a) in a serum or plasma sample of a patient known to exhibit a cancer or a placental-related pathology; and
c) comparing said presence or increase obtained in step b) to the presence or increase of said peptide or protein in a serum or plasma sample from a healthy patient.

In a preferred embodiment, the present invention relates to a method for the selection of a serum biomarker of global hypomethylation, said method comprising the steps of:
a) selecting a gene which is expressed as a protein or a peptide in a cancer and a placental cell, and whose expression in said cell is due to the global hypomethylation of the cell, particularly of the expression regulation system/promoter of said gene;
b) determining the significant presence or the increase of the protein, or a specific fragment thereof, encoded by said gene selected in step a) in a serum or plasma sample of a patient known to exhibit a cancer or a placental-related pathology, preferably compared to a healthy patient serum or plasma sample; and
c) selecting said protein, or a specific fragment thereof, encoded by said gene as a serum biomarker of global hypomethylation, whether this gene satisfy the step a) and, optionally, step b) selection.

In a more preferred embodiment, the present invention relates to a method for the selection of a serum biomarker of global hypomethylation, wherein step a) comprises selecting a gene which is expressed as a protein or a peptide in a cancer and a placental cell, and whose expression in said cell is due to the global hypomethylation of the expression regulation system of said gene.

In another particular embodiment, the present invention concerns a method for the selection of a serum biomarker of epigenetic alterations, wherein the presence or increase of said biomarker is detected at the nucleic acid level, using any method for the specific nucleic acid detection, in particular mRNA and/or cDNA, known by a person skilled in the art.

In a more particular embodiment, the present invention concerns a method for the selection of a serum biomarker of epigenetic alterations, wherein nucleic acid specifically encoding for said biomarker is quantified, by any method known by a person skilled in the art, such as Q-PCR or methods derived therefrom well known by the skilled person. In a preferred embodiment, said placental cell is a trophoblastic cell.

In an also preferred embodiment, said cancer cell is selected from tumorigenic or metastatic cancer cell.

In a preferred embodiment, said cancer is selected from breast, bladder, lung or colorectal cancer.

In another embodiment, the present invention is also directed to a method for the selection of a serum biomarker of epigenetic alterations, particularly of global hypomethylation, said method comprising the steps of:
a) selecting a gene which is expressed (more preferably protein or peptide expression) in a cancer and/or a placental cell, preferably both in a cancer cell and in a placental cell, said cell exhibiting a global hypomethylation and, optionally, the expression of said gene in said cell being due to the said global hypomethylation, particularly the hypomethylation of the regulation system/promoter of said gene expression;
b) optionally, determining the presence or the increase of the protein, or a specific fragment thereof, encoded by said gene selected in step a) in a serum or plasma sample of a patient known to exhibit a cancer or a placental-related pathology, preferably compared to a healthy patient serum or plasma sample; and
c) selecting said protein, or a specific fragment thereof, encoded by said gene as a serum biomarker of epigenetic alterations, particularly of global hypomethylation, whether this gene satisfy the step a) and, optionally, step b) selection. Methods to measure global DNA methylation or gene-specific methylation are well known by the skilled person.

Methods for DNA methylation analysis can be divided roughly into two types: global and gene-specific methylation analysis. For global methylation analysis, there are methods which measure the overall level of methyl cytosines in genome such as chromatographic methods and methyl accepting capacity assay. For gene-specific methylation analysis, a large number of techniques have been developed. Most early studies used methylation sensitive restriction enzymes to digest DNA followed by Southern detection or PCR amplification. Recently, bisulfite reaction based methods have become very popular such as methylation specific PCR (MSP), bisulfite genomic sequencing PCR. Additionally, in order to identify unknown methylation hot-spots or methylated CpG islands in the genome, several of genome-wide screen methods are also available such as Restriction Landmark Genomic Scanning for Methylation (RLGS-M), and CpG island microarray.

Among the methods which can be used for determining whether the cell exhibits a global hypomethylation or whether the gene expression regulation system or promoter is hypomethylated (see optional step b) of the above cited method), the following methods and references can be cited:
Methyl-cytosine content measure using high performance liquid chromatography (HPLC) of genomic DNA digested to individual deoxynucleosides [9, 12];
Southern blot [8];
Methyl group acceptance assay (MAA) [13];
Direct bisulphite sequencing [14] which can be adapted to assay repetitive elements using degenerate primers to conserved sequences such as LINE1 and can be accurately quantified using Pyrosequencing [20];
Methylation sensitive PCR (MSP) and quantitative PCR (qPCR) based method called MethyLight [15]; and lastly,
Monoclonal antibodies against 5-methylcytosine which have been developed and used to reliably stain methylated DNA in vitro. This has been applied to immunohistochemistry, immunofluorescence, flow cytometry and DNA immunoprecipitation combined with microarrays or high throughput sequencing [21, 22].

The term “patient” or “individual” includes mammals, e.g., humans, dogs, cows, horses, kangaroos, pigs, sheep, goats, cats, mice, rabbits, rats, and transgenic non-human animals, human is preferred.

Samples for use in the assays of the invention can be obtained by standard methods including particularly venous puncture.

In another aspect, the present invention concerns a method for diagnosing pathology associated to epigenetic alterations, particularly to global hypomethylation, of cell in a patient comprising the steps of:
i) selecting at least one serum biomarker of epigenetic alterations, particularly of global hypomethylation, selected by the method of the present invention, whose cell specific expression is due to said global hypomethylation, particularly to the hypomethylation of the system/promoter which regulates its expression;
ii) optionally, determining the methylation status, preferably the hypomethylation, of said regulation system, or part thereof, in a sample from said patient;
iii) determining the presence or the level of said biomarker selected in step a) in a serum or plasma sample of said patient,
   wherein the significant presence or the increase of said biomarker in said patient sample, preferably compared with said biomarker level found for healthy patient serum or plasma sample, is indicative of said pathology.

In a preferred embodiment, said pathology associated to epigenetic alteration is a cancer or a placental-related pathology. By “pathology associated to epigenetic alterations” it is intended to designate a pathology wherein an epigenetic alteration of the genome is observed, and particularly a global hypomethylation of the genome.

In a more particular aspect, the present invention concerns a method for diagnosing pathology associated to epigenetic alterations of cell in a patient comprising the steps of:
i) selecting at least one serum biomarker of epigenetic alterations selected by the method of the present invention, whose cell specific expression is due to said global hypomethylation,
ii) optionally, determining the methylation status in a sample from said patient;
iii) determining the presence or the level of said biomarker selected in step a) in a serum or plasma sample of said patient,

In a preferred embodiment, said placental-related pathology is selected from the group consisting of small for gestational age (SGA), intrauterine growth retardation (IUGR), pre-eclampsia and chromosomally abnormal pregnancy (such as Down syndrome, Trisomies 13 and 18, Klinefelter syndrome or XYY abnormality, preferably Down syndrome).

In a preferred embodiment, said cancer is selected from tumorigenic or metastatic cancer.

In a preferred embodiment, said cancer is selected from breast, bladder, lung or colorectal cancer.

By “placental-related pathology” it is intended to designate an anormal pregnancy linked to a pathology of the placenta.

In another aspect, the present invention is directed to a method for detecting a predisposition to, or the incidence of, placental-related pathology or cancer pathology in a patient which comprises:
i) selecting at least one serum biomarker of epigenetic alterations and, optionally, of global hypomethylation selected by a method according to the present invention;
ii) determining the presence or the level of said biomarker selected in step a) in a serum or plasma sample of said patient,
   wherein the significant presence or the increase of said biomarker according to the invention in said patient sample, preferably compared with the level of said biomarker found for healthy patient serum or plasma sample, is indicative of a predisposition to, or the incidence of, placental-related pathology or cancer.

For the detection of a biomarker at the protein level, any method for specific protein detection known by a person skilled in the art may be used. In a more particular embodiment, the present invention concerns a method for the selection of a serum biomarker of epigenetic alterations, wherein said biomarker is quantified, by any method for determining the quantity or concentration of a protein known by a person skilled in the art.

By significant increase, it is intended to mean that the level of the biomarker found in the serum or plasma sample of the patient to be tested, is at least 1.5 times, preferably twice, more preferably 3, 5 or 10 times higher than the level obtained for healthy patient.

In a preferred embodiment, in the method for detecting a predisposition to, or the incidence of, placental-related pathology or cancer pathology in a patient according to the present invention, said pathology associated to epigenetic alteration is a cancer or a placental-related pathology is selected from the group consisting of small for gestational age (SGA), intrauterine growth retardation (IUGR), pre-eclampsia and chromosomally abnormal pregnancy and/or said cancer is selected from tumorigenic or metastatic cancer, more preferably selected from breast, bladder, lung or colorectal cancer.

In a preferred embodiment, said expression regulation system is selected from promoter or retrotransposon expression regulation system.

In another aspect, the present invention is directed to a method for diagnosing pathology associated to epigenetic alterations, particularly to global hypomethylation, of cell in a patient, or to a method for detecting a predisposition to, or the incidence of, placental-related pathology or cancer pathology in a patient according to the present invention, wherein in step ii) or iii), the determination of the presence or the level of said biomarker in a serum or plasma sample of said patient is carried out by the use of antibody specifically directed against the selected serum biomarker of epigenetic alterations, particularly of global hypomethylation.

In another aspect, the present invention is directed to a method for predicting the effect of a drug targeting epigenetic modifications, and in particular the effect of a demethylating agent. According to this aspect, a method for predicting the effect of a drug targeting epigenetic modifications, particularly the effect of a demethylating agent, comprises the following steps:
i) selecting, by a method according to the invention, at least one serum biomarker of epigenetic alterations, preferably a marker of global hypomethylation,
ii) determining the presence or the level of said biomarker selected in step a) in a serum or plasma sample of said patient having being treated with said drug,
   wherein the significant presence or the increase of said biomarker in said patient sample, compared with the level of said biomarker in a serum or plasma sample of said patient before said treatment, is indicative of the effect of said drug.

In another aspect, the present invention is directed to a method for following the effect of a drug targeting epigenetic modifications, and in particular the effect of a demethylating agent. According to this aspect, a method for following the effect of a drug targeting epigenetic modifications, particularly the effect of a demethylating agent, comprises the following steps:
i) selecting, by a method according to the invention, at least one serum biomarker of epigenetic alterations, preferably a marker of global hypomethylation,
ii) determining the presence or the level of said biomarker selected in step a) in a serum or plasma sample of said patient, said sample being collected at at least two different times of said treatment, possibly before, during and/or after the treatment.

In a particular aspect, the present invention is directed to a method for predicting the effect of a drug targeting epigenetic modifications, and in particular the effect of a demethylating agent, wherein said at least one serum biomarker of epigenetic alterations, particularly of global hypomethylation, is selected from the group consisting of the INSL4 (pro-EPIL) and hCGβ type II peptide (or protein) biomarker.

In another particular aspect, the present invention is directed to a method for following the effect of a drug targeting epigenetic modifications and in particular the effect of a demethylating agent, wherein said at least one serum biomarker of epigenetic alterations, particularly of global hypomethylation, is selected from the group consisting of the INSL4 (pro-EPIL) and hCGβ type II peptide (or protein) biomarker.

In a particular aspect, the present invention is directed to a method for diagnosing pathology associated to epigenetic alterations, particularly to global hypomethylation, of cell in a patient, or to a method for detecting a predisposition to, or the incidence of, placental-related pathology or cancer pathology in a patient according to the present invention, wherein said at least one serum biomarker of epigenetic alterations, particularly of global hypomethylation, is selected from the group consisting of the INSL4 (pro-EPIL) and hCGβ type II peptide (or protein) biomarker.

In the present description, peptide, polypeptide or protein are used interchangeably and have the same meaning

EPIL is a 139-amino-acid polypeptide which is synthesized as a preprohormone characterized by a signal peptide, a B-chain, a connecting C-peptide and a terminal A-chain. In placenta, it was found that trophoblast cells translate INSL4 mRNAs into immunoreactive pro-EPIL peptides comprising the B-, C- and A-chains (see FIG. 1). Initially, pro-EPIL peptide was detected in amniotic fluid and maternal serum during normal pregnancy.

The amino-acid and mRNA sequence of the human Early placental insulin-like protein (INSL4 or EPIL, see GenPep accession number NP 002186) is: Complete pro-EPIL amino acids sequence (SEQ ID NO: 1)

| | |
|---|---|
| 1 | MASLFRSYLP AIWLLLSQLL RESLAAELRG CGPRFGKHLL |
| | SYCPMPEKTF |
| 51 | TTTPGGWLLE SGRPKEMVST SNNKDGQALG TTSEFIPNLS |
| | PELKKPLSEG |
| 101 | QPSLKKIILS RKKRSGRHRF DPFCCEVICD DGTSVKLCT. |

Respectively, amino acids 1-22 (aa1-22) correspond to the signal peptide (SEQ ID NO: 2); aa 23-52 to the “B chain” (SEQ ID NO: 3); aa 59-108 to the “C chain (SEQ ID NO: 4) and aa 115-139 to the “A chain” (SEQ ID NO: 5).

The sequence of Insulin-like 4 (placenta) (INSL4) mRNA (see Genbank accession number NM 002195) (SEQ ID NO: 6) is the following:

| | |
|---|---|
| 1 | agtctggagc ccagaaggga cacaccagca cagtctggta |
| | ggctacagca gcaagtctct |
| 61 | aaagaaaggc tgagaacacc cagaacagga gagttcaggt |
| | ccaggatggc cagcctgttc |
| 121 | cggtcctatc tgccagcaat ctggctgctg ctgagccaac |
| | tccttagaga aagcctagca |
| 181 | gcagagctga ggggatgtgg tccccgattt ggaaaacact |
| | tgctgtcata ttgccccatg |
| 241 | cctgagaaga cattcaccac caccccagga gggtggctgc |
| | tggaatctgg acgtcccaaa |
| 301 | gaaatggtgt caacctccaa caacaaagat ggacaagcct |
| | taggtacgac atcagaattc |
| 361 | attcctaatt tgtcaccaga gctgaagaaa ccactgtctg |
| | aagggcagcc atcattgaag |
| 421 | aaaataatac tttcccgcaa aaagagaagt ggacgtcaca |
| | gatttgatcc attctgttgt |
| 481 | gaagtaattt gtgacgatgg aacttcagtt aaattatgta |
| | catagtagag taatcatgga |
| 541 | ctggacatct catccattct catatgtatt ctcaatgaca |
| | aattcactga tgcccaatta |
| 601 | aatgattgct gtttaaa |

Respectively, the sequence nt106-522 correspond to the pro-EPIL coding sequence (SEQ ID NO: 7); nt106-171 to the signal peptide (SEQ ID NO: 8); nt172-261 to the “B chain” (SEQ ID NO: 9); nt280-429 to the “C chain (SEQ ID NO: 10); and nt448-522 to the “A chain” (SEQ ID NO: 11).

INSL4 gene encodes a precursor that undergoes post-translational cleavage to produce 3 polypeptide chains, A-C, that form tertiary structures composed of either all three chains, or just the A and B chains (see European patent document Bellet et al., published under the number EP 2 281 195 B1 on Feb. 22, 2012).

It shall be understood that the term “specific peptide fragment” designates in particular a fragment of an amino acid sequence of a polypeptide having at least one of the functional characteristics or properties of the complete polypeptide, notably in that it is capable of being recognized by a specific antibody and/or that the expression level of such a specific peptide fragment is correlated to expression level of the complete or partial pro-EPIL expressed.

It is understood that the term “specific peptide fragment” designates particularly a polypeptide including a minimum of 9 amino acids, preferably 10, 11 or 12 amino acids, and most preferably 15, 20 or 25 amino acids of the sequence SEQ ID No: 1, preferably this fragment contains a fragment of at least the chain A, B or C of the human pro-EPIL.

Specific anti-pro-EPIL monoclonal or polyclonal antibodies are available to the skilled man. An isolated pro-EPIL, or a specific fragment thereof, can be used as an immunogen to generate antibodies that bind such protein using standard techniques for polyclonal and monoclonal antibody preparation. It may be also possible to use any fragment of these protein which contains at least one antigenic determinant may be used to generate these specific antibodies.

A protein immunogen typically is used to prepare antibodies by immunizing a suitable subject, (e.g., rabbit, goat, mouse or other mammal) with the immunogen. An appropriate immunogenic preparation can contain said pro-EPIL polypeptide, or fragment thereof, and further can include an adjuvant, such as Freund's complete or incomplete adjuvant, or similar immuno-stimulatory agent.

Monoclonal antibody (mAb) directed specifically (capable of binding) to the EPIL C-chain 98-108 region, such EPIL15 mAB (preferably as capture antibody) and/or mAb directed specifically (capable of binding) to the EPIL A-chain 125-137, such a EPIL02 (preferably as tracer) are more preferred.

The alpha subunit of human Chorionic Gonadotropin (hCG), which is a member of the glycoprotein hormone family, is encoded by one gene on chromosome 12₈21.1-23. The hCGβ subunit however, is encoded by six non-allelic genes (CGB genes). The sequencing of the human genome offers a novel opportunity to check the sequences and organization of these genes clustered on chromosome 19q13.3 and named CGB1 or β1, CGB2 or β2, CGB3 or β3, CGB5 or β5 (SEQ ID No: 12 and SEQ ID No. 13), CGB7 or β7 and CGB8 or β8. Genes β1 and β2 are considered pseudogenes that are not expressed whereas the remaining four genes encode the same protein, with the exception of β7 gene which encodes an alanine at position 117 as opposed to an aspartic acid in the other three genes. On the basis of the amino acid residues displayed at position 117, genes encoding the hCGβ subunit were classified as type I genes if they encoded an alanine (β7) or as type II genes if they encoded an aspartic acid (β3, β5, β8) [23]

In another particular aspect, the present invention is directed to a method for diagnosing pathology associated to epigenetic alteration and, optionally, to global hypomethylation, of cell in a patient, said method comprises the step of:
a) specifically detecting or quantifying the presence of the pro-EPIL peptide and/or of hCGβ type II subunit in a serum or plasma sample from said patient susceptible of containing pro-EPIL and/or hCGβ subunits type II, by a method which implements the use of a polyclonal or a monoclonal antibody (mAb) specifically directed to said pro-EPIL peptide, or to a specific fragment thereof, and/or pro-EPIL peptide and/or the use of a polyclonal antibody or mAb specifically directed to said hCGβ type II subunit, or to a specific fragment thereof.

In another particular aspect, the present invention is directed to a method for detecting a predisposition to, or the incidence of, placental-related pathology or cancer pathology in a patient, said method comprises the step of:
a) specifically detecting or quantifying the presence of the pro-EPIL peptide and/or of hCGβ type II subunit peptide in a serum or plasma sample from said patient susceptible of containing pro-EPIL and/or hCGβ subunits type II peptide, by a method which implements the use of a polyclonal antibody or a mAb specifically directed to said pro-EPIL peptide, or to a specific fragment thereof, and/or the use of a polyclonal antibody or a mAb specifically directed to said hCGβ type II subunit, or to a specific fragment thereof.

In a preferred embodiment, said antibody specifically directed to said pro-EPIL peptide or to said hCGβ type II subunit peptide is selected from the group consisting of:
a) polyclonal, monoclonal chimeric or humanized antibodies able to selectively bind, or which selectively bind to an epitope-containing a pro-EPIL polypeptide comprising a contiguous span of at least 9 to 10 amino acids of a pro-EPIL fragment, particularly of a fragment of at least the chain A, B or C of the human pro-EPIL; or
b) polyclonal, monoclonal chimeric or humanized antibodies able to selectively bind hCGβ type II subunit, or a specific fragment thereof, preferably able to selectively bind a discontinuous epitope that comprises region 1-7 with a lysine and a proline residue at position 2 and 4 respectively and region 82-92 of hCGβ, or an anti hCGβ type II-mAb specific binding fragment thereof.

In a more preferred embodiment, this antibody specifically directed to said hCGβ type II subunit peptide is selected from the group consisting of:
a monoclonal antibody (mAb) specifically directed to a discontinuous epitope that comprises region 1-7 with a lysine and a proline residue at position 2 and 4 respectively (SEQ ID No. 14) and region 82-92 of hCGβ,
a mAb specifically directed to a discontinuous epitope that comprises region 1-7 with a lysine and a proline residue at position 2 and 4 respectively and region 82-92 of hCGβ, wherein this antibody is produced by an hybridoma obtained from a mouse which has been prior immunized with an antigen comprising at least the fragments 1-7 with a lysine and a proline residue at position 2 and 4 respectively and 82-92 of hCGβ, said hybridoma being selected based on the capability of its secreted mAb to specifically recognizing the fragments 1-7 with a lysine and a proline residue at position 2 and 4 of the hCGβ type II;
the mAb FBT-11-II produced by the hybridoma deposited with the CNCM (Collection Nationale de Cultures de Microorganismes, Institut Pasteur, 25 rue du Docteur Roux, F-75724 PARIS Cedex 15) on Mar. 9, 2010 under the number 1-4281;
the mAb FBT-11 produced by the hybridoma deposited with the CNCM on Oct. 3, 1985 under the number 1-489;
a recombinant mAb having a sequence comprising at least the 6 CDRs (Complementary Determining Region) of the mAb FBT-11-II produced by the hybridoma deposited under the number I-4281or at least the 6 CDRs of the the mAb FBT-11 produced by the hybridoma deposited under the number 1-489; and
a hCGβ type II-binding fragment thereof.

In the method of the present invention, the determination of the presence or the level of said biomarker in a serum or plasma sample of said patient is carried out preferably by immunoassay, more preferably by ELISA immunoassay or radioimmunoassay in blood serum or plasma.

When the presence or the increase of hCGβ subunits type II biomarker is desired to be determined in a serum or plasma sample, in a preferred embodiment of the the present invention this method comprises the steps of:
a) contacting the biological sample from the patient to be tested or from the control with an anti-hCGβ subunits type II antibody selected from the group as described above or a hCGβ type II-binding fragment thereof,
   preferably under conditions permitting the binding of said antibody to the hCGβ type II subunits present in said biological sample; and
b) measuring the amount of the complex formed between said antibody bound to the hCGβ type II subunits so as to thereby determine the amount of hCGβ type II in the sample.

In a preferred embodiment of the present invention, said method for specifically detecting or quantifying the presence of hCGβ type II subunits in a biological sample comprises the steps of:
a) contacting the biological sample from the subject with a capture antibody capable of binding hCGβ type I and type II under conditions permitting the formation of a complex between the antibody and any hCGβ present in the sample;
b) contacting the complex formed with a second antibody (tracer antibody) which is a specific anti-hCGβ subunits type II antibody selected from the group as described above or a hCGβ type II-binding fragment thereof,
   preferably under conditions permitting the binding of said antibody to the hCGβ type II subunits present in said biological sample; and
c) measuring the amount of the second antibody bound to the complex formed so as to thereby determine the amount of hCGβ type II in the sample.

According to the present invention and in a more preferred embodiment,
in step a), the capture antibody is bound to a solid support and the step comprises the removing of any unbound sample from the solid support; and
in step b), the solid support is contacted with the second antibody.

In a more preferred embodiment, the first antibodies used (capture antibody) are mAbs directed to the carboxyl terminal portion of hCGβ, preferably directed to an epitope comprising at least 6 amino acid residues, preferably at least 10, 12, 15 or 20 residues between the fragment AA118-147 of the hCGβ type I or II, and/or preferably, also directed to an epitope comprising at least 6 amino acid residues, preferably at least 10, 12, or 15 residues between the fragment AA95-116 of the hCGβ type I or II preferably the monoclonal antibodies (mAbs) named FB09 or FB12 which were obtained as referenced in the Examples.

The antibody, named FBT10, secreted by the hybridoma deposited under the number 1-488 with the CNCM on Oct. 3, 1985 can be also used as capture antibody in the method of the present invention.

In a more preferred embodiment, the antibodies anti-pro-EPIL or anti-hCGβ type II used, particularly can be labelled antibody.

“Labelled antibody” as used herein includes antibodies that are labeled by a detectable means and includes enzymatically, radioactively, fluorescently, chemiluminescently, bioluminescently, biotin or magnetic bead labeled antibodies by any of the many different methods known to those skilled in this art.

One of the ways in which an antibody can be detectably labelled is by linking the same antibody to an enzyme. This enzyme, in turn, when later exposed to its substrate, will react with the substrate in such a manner as to produce a chemical moiety which can be detected, for example, by spectrophotometric, fluorometric or by visual means. Enzymes which can be used to detectably label pro-EPIL or hCGβ type II-specific antibody include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

Detection or quantification may be accomplished using any of a variety of immunoassays. For example, by radioactively labelling an antibody, it is possible to detect the antibody through the use of radioimmune assays. A description of a radioimmune assay (RIA) may be found in Laboratory Techniques and Biochemistry in Molecular Biology, by Work T. S. et al., North Holland Publishing Company, NY (1978), with particular reference to the chapter entitled “An Introduction to Radioimmune Assay and Related Techniques” by Chard T. The radioactive isotope can be detected by such means as the use of a gamma counter or a scintillation counter or by audioradiography. Isotopes which are particularly useful for the purpose of the present invention are: ³H, ¹³¹I, ₃₅S, ¹⁴C, and preferably ¹²⁵I.

It is also possible to label an antibody with a fluorescent compound. When the fluorescently labelled antibody is exposed to light of the proper wave length, its presence can then be detected due to fluorescence. Among the most commonly used fluorescent labelling compounds are fluorescein isothiocyanate, rhodamine, phycoerytherin, phycocyanin, allophycocyanin, ophthaldehyde and fluorescamine.

An antibody can also be detectably labelled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody can also be detectably labelled by coupling it to a chemiluminescent compound. The presence of the chemiluminescent-tagged antibody is then determined by detecting the presence of luminescence that arises during the course of a chemical reaction. Examples of particularly useful chemiluminescent labelling compounds are luminol, luciferin, isoluminol, theromatic acridinium ester, imidazole, acridinium salt and oxalate ester.

Likewise, a bioluminescent compound may be used to label an antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labelling are luciferin, luciferase and aequorin.

In the detection or quantification assays implemented in the method of the invention, the amount of binding of the antibody to the biological sample can be determined by the intensity of the signal emitted by the labelled antibody and/or by the number cells in the biological sample bound to the labelled antibody.

The detection or the level of the selected serum biomarker of epigenetic alterations, particularly of global hypomethylation, such as pro-EPIL or hCGβ type II biomarker, in a biological sample may be determined by a radioimmunoassay, an immunoradiometric assay, and/or an enzyme immunoassay.

“Radioimmunoassay” is a technique for detecting and measuring the concentration of an antigen using a labelled (i.e. radioactively labelled) form of the antigen (hCGβ type II). The concentration of hCGβ type II in a biological sample is measured by having the antigen in the sample compete with a labelled (i.e. radioactively) antigen for binding to an antibody to the antigen. To ensure competitive binding between the labelled antigen and the unlabeled antigen, the labelled antigen is present in a sufficient concentration to saturate the binding sites of the antibody. The higher the concentration of antigen in the sample, the lower the concentration of labelled antigen that will bind to the antibody.

In a radioimmunoassay, to determine the concentration of labelled antigen bound to an antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (i.e. covalently) to Sepharose beads, polystyrene wells, polyvinylchloride wells, or microtiter wells. By comparing the concentration of labelled antigen bound to antibody to a standard curve based on samples having a known concentration of antigen, the concentration of antigen in the biological sample can be determined.

An “Immunoradiometric assay” (IRMA) is an immunoassay in which the antibody reagent is radioactively labeled. An IRMA requires the production of a multivalent antigen conjugate by techniques such as conjugation to a protein e.g., rabbit serum albumin (RSA). The multivalent antigen conjugate must have at least 2 epitopes per molecule and these epitopes must be located at a sufficient distance to allow binding by at least two antibodies to the antigen. For example, in an IRMA the multivalent antigen conjugate can be attached to a solid surface such as a plastic sphere.

Unlabelled “sample” antigen and antibody to antigen which is radioactively labelled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA)”. The “Enzyme-Linked Immunosorbent Assay (ELISA)” is a technique for detecting and measuring the concentration of an antigen using a labelled (i.e. enzyme linked) form of the antibody.

In a “sandwich ELISA”, an antibody (i.e. anti-pro-EPIL or anti- hCGβ) is linked to a solid phase (i.e. a microtiter plate) and exposed to a biological sample containing antigen (i.e. pro-EPIL or hCGβ type II). The solid phase is then washed to remove unbound antigen. A labelled antibody (i.e. anti-pro-EPIL or anti-hCGβ type II enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and 3-galactosidase. The enzyme linked antibody reacts with a substrate to generate a colored reaction product that can be assayed for.

In a “competitive ELISA”, antibody is incubated with a sample containing antigen (i.e. hCGβ type II). The antigen-antibody mixture is then contacted with an antigen-coated solid phase (i.e. a microtiter plate). The more antigen present in the sample, the less free antibody that will be available to bind to the solid phase. A labelled (i.e. enzyme linked) secondary antibody is then added to the solid phase to determine the amount of primary antibody bound to the solid phase.

In another aspect, the present invention is directed to a kit for diagnosing a pathology associated to epigenetic alteration, particularly to global hypomethylation, of cell in a patient, said kit comprising:
a) an antibody directed specifically against the pro-EPIL peptide, or specific fragment thereof; or/and
b) an antibody directed specifically against the hCGβ-type II subunit peptide, or specific fragment thereof.

In a preferred embodiment, said pathology associated to epigenetic alterations is a cancer or a placental-related pathology. Preferably, said pathology associated to epigenetic alterations is a placental-related pathology is selected from the group consisting of small for gestational age (SGA), intrauterine growth retardation (IUGR) and pre-eclampsia and/or said cancer is selected from tumorigenic or metastatic cancer, more preferably selected from breast, bladder, colorectal or lung cancer.

In another aspect, the present invention is directed to a kit for detecting a predisposition to, or the incidence of, placental-related pathology or cancer pathology in a patient, said kit comprising:
a) an antibody directed specifically against the pro-EPIL peptide, or a binding fragment thereof, optionally labelled; or/and
b) an antibody directed specifically against the hCGβ-type II subunit peptide, or a binding fragment thereof, optionally labelled.

In another aspect, the present invention is directed to a kit for diagnosing a pathology associated to epigenetic alteration of cell in a patient or for detecting a predisposition to, or the incidence of, a placental-related pathology or cancer pathology in a patient, said kit comprising:
a) an antibody directed specifically against the pro-EPIL peptide, or specific fragment thereof; and
b) an antibody directed specifically against the hCGβ-type II subunit peptide, or specific fragment thereof.

In a preferred embodiment, said antibody specifically directed to said pro-EPIL peptide or to said hCGβ type II subunit peptide contained in the kit of the present invention is selected from the group consisting of:
a) polyclonal, monoclonal chimeric or humanized antibodies able to selectively bind, or which selectively bind to an epitope-containing a pro-EPIL polypeptide comprising a contiguous span of at least 9 to 10 amino acids of a pro-EPIL fragment, particularly of a fragment of at least the chain A, B or C of the human pro-EPIL; or
b) polyclonal, monoclonal chimeric or humanized antibodies able to selectively bind hCGβ type II subunit, or a specific fragment thereof, preferably able to selectively bind a discontinuous epitope that comprises region 1-7 with a lysine and a proline residue at position 2 and 4 respectively and region 82-92 of hCGβ, or an anti hCGβ type II-mAb specific binding fragment thereof.

In a more preferred embodiment, said antibody specifically directed to hCGβ type II subunit peptide is selected from the group consisting of:
a monoclonal antibody (mAb) specifically directed to a discontinuous epitope that comprises region 1-7 with a lysine and a proline residue at position 2 and 4 respectively and region 82-92 of hCGβ,
a mAb specifically directed to a discontinuous epitope that comprises region 1-7 with a lysine and a proline residue at position 2 and 4 respectively and region 82-92 of hCGβ, wherein this antibody is produced by an hybridoma obtained from a mouse which has been prior immunized with an antigen comprising at least the fragments 1-7 with a lysine and a proline residue at position 2 and 4 respectively and 82-92 of hCGβ, said hybridoma being selected based on the capability of its secreted mAb to specifically recognizing the fragments 1-7 with a lysine and a proline residue at position 2 and 4 of the hCGβ type II;
the mAb FBT-11-II produced by the hybridoma deposited with the CNCM (Collection Nationale de Cultures de Microorganismes, Institut Pasteur, 25 rue du Docteur Roux, F-75724 PARIS Cedex 15) on Mar. 9, 2010 under the number 1-4281;
the mAb FBT-11 produced by the hybridoma deposited with the CNCM on Oct. 3, 1985 under the number 1-489;
a recombinant mAb having a sequence comprising at least the 6 CDRs (Complementary Determining Region) of the mAb FBT-11-II produced by the hybridoma deposited under the number 1-4281 or at least the 6 CDRs of the the mAb FBT-11 produced by the hybridoma deposited under the number 1-489; and
a hCGβ type II-binding fragment thereof.

In another particular embodiment, the present invention relates to a kit for detecting a predisposition to, or the incidence of, placental-related pathology or cancer pathology in a patient, said kit comprising:
a) an antibody directed specifically against the pro-EPIL peptide, or specific fragment thereof; and
b) an antibody directed specifically against the hCGβ-type II subunit peptide, or specific fragment thereof.

In another particular embodiment, the present invention relates to the use of a kit according to the invention for diagnosing a pathology associated to epigenetic alteration of cells, for detecting a predisposition to placental-related pathology or cancer pathology or for detecting the incidence of placental-related pathology or cancer pathology in a patient.

The following examples and figures are given for the purpose of illustrating various embodiments of the invention and are not meant to limit the present invention in any fashion.

### LEGENDS OF THE FIGURES

FIG. 1: Schematic representation of the primary structure of pro-EPIL peptide and localization of antibody binding sites recognized by monoclonal antibodies EPIL15 and EPIL02. Amino acid residues are indicated by one-letter code.

FIG. 2: Organization of the CGβ/LHβ gene cluster and amino acid sequences of expressed genes. Only genes CGβ3, CGβ5, CGβ7 and CGβ8 code for the hCGβ subunit. Type I genes code for a mature protein with an arginine residue at amino acid 2, a methionine residue at amino acid 4 (see SEQ ID No. 15) and an alanine residue at amino acid 117. Type II genes encode a mature protein with a lysine residue at amino acid 2, a proline residue at amino acid 4 and an aspartic acid at amino acid 117.

FIG. 3: Curve of viability of a cell line treated with 5-Azacytidine (5-Aza-dC) at increasing doses of 5-Aza-dC. The percentage of viability is expressed relatively to the concentration of 5-Aza-dC (in microM), the doses A and B correspond respectively to the 5-Aza-dC concentrations at the inflexion point and at the 50% inhibition of cell viability,

FIGS. 4A and 4B: FIG. 4A is a histogram representation of the pro-EPIL supernatant level (in ng/millions of cells) in different tissues. FIG. 4B is a histogram representation of the hCG beta type II supernatant level (in ng/millions of cells) in different tissues.

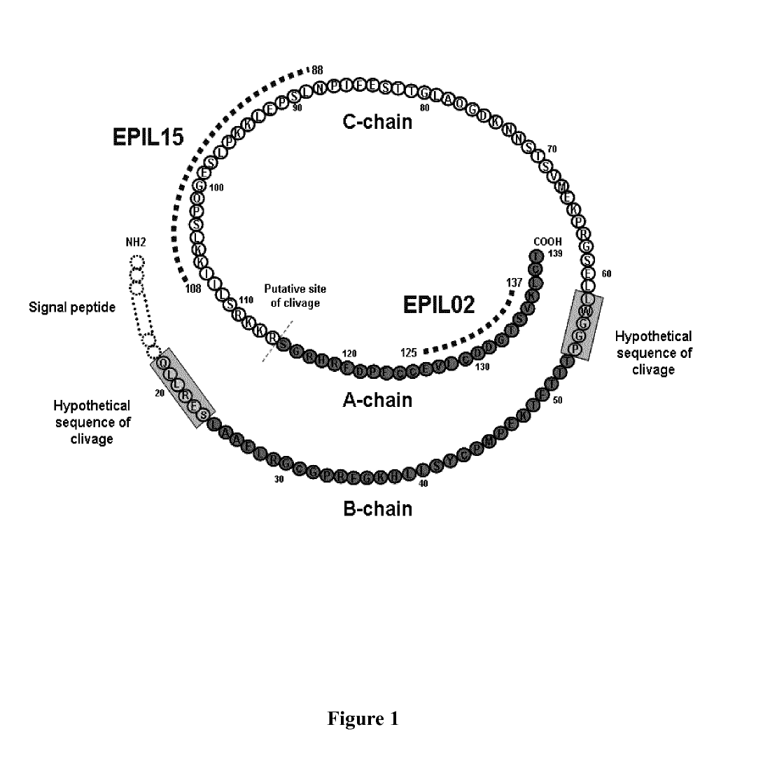

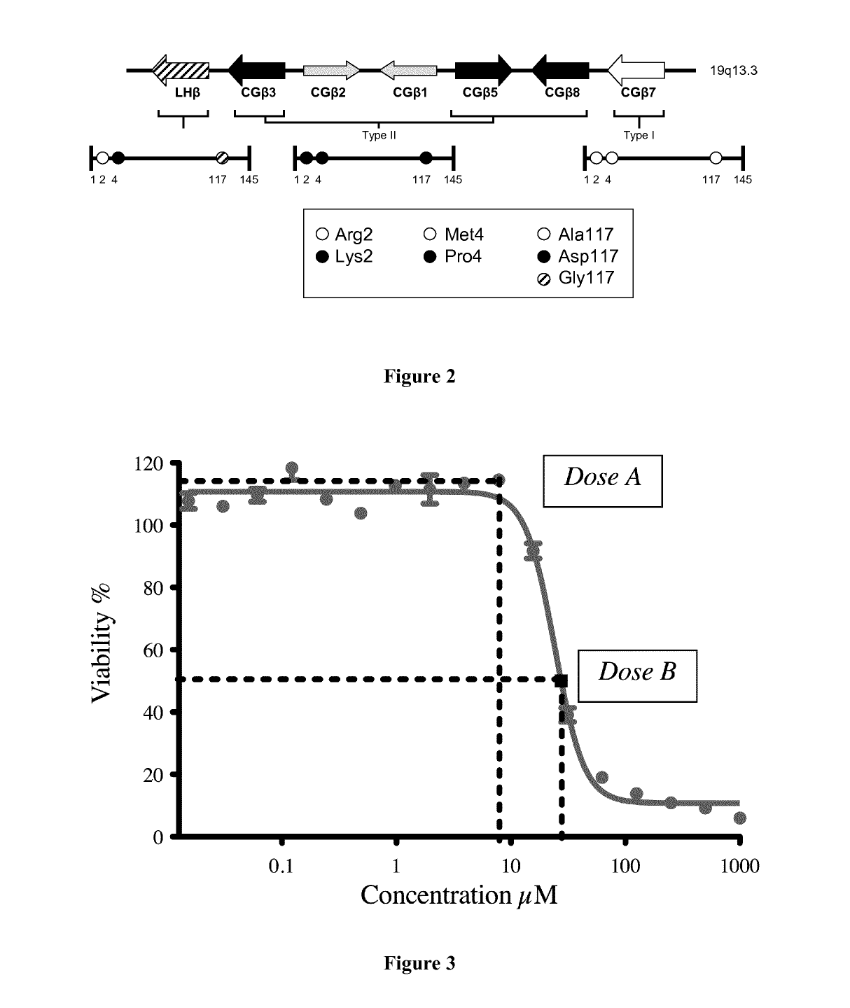

### EXAMPLE 1

### Materials and Methods

See International patent application Bellet et al., filed under N° EP2011/053676 on Mar. 11, 2011 and published under number WO2011/110657.

### Monoclonal Antibodies

Monoclonal antibody (mAb) EPIL15 is directed to the EPIL C-chain 98-108 region. This antibody serves as capture antibody on a solid phase support, while biotinylated mAb EPIL02 directed to the EPIL A-chain 125-137 region is used as tracer. The standard curve was constructed with a peptide spanning the 76-139 portion of pro-EPIL used at increasing concentrations ranging from 0.7 ng/mL to 100 ng/mL. Linearity in a range of 0.7 ng/mL to 100 ng/mL was consistently shown in between and within-run assays. The sensitivity of this ELISA is 1 ng/mL (see European patent document, Bellet et al., published under the number EP 2 281 195 B1 on Feb. 22, 2012).

Monoclonal antibody (mAbs) FBT-11-II is an IgG1-Kappa produced by the hybridoma deposited pursuant to, and in satisfaction of, the requirements of the Budapest Treaty on the International Recognition of the Deposit of Microorganisms for the Purposes of Patent Procedure with the CNCM (Collection Nationale de Cultures de Microorganismes, Institut Pasteur, 25 rue du Docteur Roux, F-75724 PARIS Cédex 15) on Mar. 9, 2010 under the number 1-4281.

The hybridoma 1-4281 which secretes the FBT-11-II antibody results from successive subcloning cycles of the the hybridoma 1-489 also deposited pursuant to and in satisfaction of, the requirements of the Budapest Treaty on the International Recognition of the Deposit of Microorganisms for the Purposes of Patent Procedure with the CNCM on Oct. 3, 1985.
Monoclonal antibodies (mAbs) FB09, FB12 and FBT11-II were obtained as previously described (see International patent application filed under N° EP2011/053676 on 11 Mar. 2011 and published under number WO2011/110657 for references).

MAbs FB09 and FB12, elicited against a synthetic peptide analogous to the COOH 109-145 terminal portion (CTP) of hGGβ, are directed against the 134-139 and 110-116 regions, respectively [24]. These mAbs are specific for either hCG or its hCGβ subunit and do not bind to LH or its LHβ subunit. MAb FBT11 and FBT-11-II elicited against purified hCGβ subunit (CR 129) are directed to a discontinuous epitope that comprises region 1-7, with a lysine and a proline residue at position 2 and 4 respectively, and region 82-92 of hCGβ. FBT-11 and FBT-11-II are specific for the hCGβ subunit and do not bind to hCG, LH or its LHβ subunit [25].

Two-Site ELISA (Measure of the hCGβ Type II Level)

Maxisorp nunc plates (Thermo Fisher Scientific, Brebières, France) were coated with 0.25 μg of monoclonal antibody FB09 and/or 0.25 μg of monoclonal antibody FB12 in 0.1M phosphate buffer pH 7.4, blocked with 1% bovine serum albumin in PBS and incubated with the hCGβ standards (ELSA-FBhCG from CIS Bio International, France; 1 ng CIS=1 mIU 1^{st} IRP WHO 75/551) for 1 h at 37° C. Bound hCGβ was detected with monoclonal antibody FBT11-II coupled with biotin for 1 h at 37° C. (Biotin Labeling Kit —NH₂ from Interchim, Montluçon, France). The plate was then incubated with Immunopure streptavidin Horseradish peroxydase conjugated (Pierce, Thermo Fisher Scientific, Brebières, France) for 10 min at room temperature (RT). TMB from Pierce was used as the substrate and the absorbance was read at 450 nm. Experiments were done in duplicate. The standard curve was constructed with the hCGβ standards used at increasing concentrations ranging from 0.21-47 ng/ml. Linearity was consistently shown in between run assays.

It is known that FBT11-II specifically recognizes hCGβ encoded by type II genes at the cellular level and FBT11-II recognizes hCGβ encoded by type II genes and produced by trophoblasts during the course of gestation.

It has already be concluded that FBT11 and FBT11-II are directed against a highly specific and discontinuous epitope that comprises region 1-7 with a lysine and a proline residue at position 2 and 4 respectively and region 82-92 of hCGβ (see International patent application WO 2011/110657)

ELISA Specific for hCGβ Encoded by Type II Genes

It has been also demonstrated in that cited patent document filed under N° EP2011/053676 on Mar. 11, 2011 and published as WO 2011/110657, that enhanced binding of indicator antibody to hCG was obtained using anti-CTP antibodies FB09 and FB12 as capture antibodies. This assay could be useful to determine the presence or absence and the level of hCGβ encoded by type II genes in biological fluids such as serum or plasma.

### EXAMPLE II

### Presence in the Serum of Cancer Patients of Pro-EPIL That is Expressed as a Consequence of Epigenetic Alterations in Both Placental and Cancer Cells, Pro-EPIL Peptide as Surrogate Biomarker of Both Epigenetic Alterations, Particularly of Global Hypomethylation

The first biomarker, pro-EPIL is a peptide encoded by INSL4 gene [26, 27]. In normal tissues, INSL4 is expressed strongly only in placenta. Much lower expression (˜10,000 times lower than the placenta) is detected in thymus, testis breast, stomach and uterus [28]. The other normal tissues contained very little or no detectable INSL4 mRNA. The placenta specific expression of INSL4 is mediated by the 3′ LTR of the HERV element inserted in the INSL4 promoter. This placenta specific expression via HERV element integration could contribute to the aggressive growth phenotype of normal placenta. Moreover, INSL4 gene is also expressed by trophoblastic and nontrophoblastic cancer cells and may play a role in the invasive properties of malignant cells [29]. Interestingly, amongst genes which have a placenta specific expression due to hypomethylation of the retrotransposons that regulate their expression, the INSL4 gene has the advantage of being transcribed and translated into a well-identified peptide designated as pro-EPIL. This peptide can be detected in biological fluids using a two-site immunoassay based on monoclonal antibodies and is present in detectable amount in the serum [30].

### EXAMPLE III

### Presence in the Serum of Cancer Patients of Free hCGβ Type II That is Expressed as a Consequence of Epigenetic Alterations in Both Placental and Cancer Cells, free hCGβ Type II as Surrogate Biomarker of Both Epigenetic Alterations and Global Hypomethylation

This second biomarker, human chorionic gonadotropin beta subunit type II (free hCGβ type II), is a protein encoded by type II CGB genes CGB3, CGB5 and CGB8.The expression of these type II CGB genes is upregulated in both trophoblastic and neoplastic cells while type I CGB gene CGB7 which encodes free hCGβ type I is expressed in many normal cells [23]. In placenta, it has been shown that the entire CGB locus appears to be very undermethylated, leading to the expression of type II CGB genes at very high levels. Moreover, recent observations demonstrate that the CpG sites mapping to the promoters of type II CGB genes are specifically less methylated in cytotrophoblasts (placental cells) in comparison to fibroblasts [31]. Thus, the most likely explanation for the transcription of type II CGB genes in neoplastic cells of differing histological origin might be that demethylation of critical sites plays a major role in the transcriptional activation of type II CGB genes. The free hCGβ type II is detected in the serum of patients with trophoblastic and non-trophoblastic cancers of various histological origin. Until recently, it has been difficult to prove that a sensitive immunoassay assays would be capable of specifically measuring the presence of free hCGβ type II in the serum without detecting free hCGβ type I. However, it was found recently that both FBT11 and FBT11-II were capable of specifically recognizing the free hCGβ type II thanks to its binding to antigenic sites that comprises two amino acid residues only present on free hCGβ type II and absent from free hCGβ type I. Thus, the hypomethylated state of type II CGB genes in both placenta and cancer and the possibility of specifically detecting the protein products of these genes in serum lead to the selection of free hCGβ type II as a protein biomarker that can complement pro-EPIL as surrogate biomarkers of epigenetic alterations and global hypomethylation, a specific feature of cancer cells.

### EXAMPLE IV

### Results

The levels of pro-EPIL and free hCGβ type II was determined in the serum of patients with lung, bladder or colorectal cancers using specific two-site immunoassays based on monoclonal antibodies. The first immunoassay is based on monoclonal antibody (mAb) EPIL15 as capture antibody and mAb EPIL 02 as tracer for measuring pro-EPIL, while the sandwich immunoassay utilized for measuring free hCGβ type II is based on mAb FBT11-II. The mAb FB09 and FB12 are used to capture the antigen. The assay for pro-EPIL has a functional sensitivity of 1 ng/ml while the functional sensitivity of the assay for free hCGβ type II is 0.1 ng/ml. Serum levels of pro-EPIL were higher than 1 ng/ml in 2 out of 100 apparently healthy women and in 3 out of 100 apparently healthy men.

Serum levels of free hCGβ type II were consistently lower than 0.1 ng/ml in our series of 100 apparently healthy women and 100 apparently healthy men. In a series of 129 patients with lung cancer, 28 (21.7%) patients had increased serum levels of pro-EPIL (>1 ng/ml), 35 (27%) patients had increased serum levels of hCGβ type II (>0.1ng/m1). Indeed, 54 (41.8%) patients with lung cancers display increased serum levels of either pro-EPIL, free hCGβ type II or both. In a series of 132 patients with colorectal cancer, 9 (6.8%) patients had increased serum levels of pro-EPIL (>1 ng/ml), 14 (10.6%) patients had increased serum levels of hCGβ type II (>0.1ng/m1). Indeed, 23 (17.4%) patients with colorectal cancers display increased serum levels of either pro-EPIL or free hCGβ type II. In a series of 10 patients with bladder cancer, 1 (10%) had increased serum levels of pro-EPIL (>1 ng/ml), 3 (30%) had increased serum levels of hCGβ type II (>0.1 ng/m1). Indeed, 4 (40%) patients with bladder cancers display increased serum levels of either pro-EPIL or free hCGβ type II.

### EXAMPLE V

### Determination of Pro-EPIL and hCGβ Type II in the Supernatant of Non Trophoblastic Cancer Cell Lines

The production of pro-EPIL and hCGβ type II was determined in the supernatant of 33 non-trophoblastic cancer cell lines established from bladder (n=15), breast (n=2), colon (n=8) and lung (n=8) tumors. In addition, pro-EPIL and hCGβ type II levels were also measured in the supernatant of JAR and JEG-3, two trophoblastic cell lines derived from placental choriocarcinomas. Results are presented in the following Table 1.

TABLE 1

| | | Pro-EPIL | hCG type II |
|---|---|---|---|
| | | (ng/million of | (ng/million of |
| Organ | Cell line | cells) | cells) |
| Bladder | CHA | 0 | 2.29 |
| | HT1197 | 48.2 | 15.32 |
| | HT1376 | 315.4 | 0.2 |
| | HTB9 | 0 | 28.09 |
| | J82 | 0 | 20.9 |
| | OBR | 2.08 | 3.5 |
| | PEL | 0 | 70.17 |
| | RT112 | 0 | 38.87 |
| | RT4 | 0 | 1.23 |
| | SCABER | 0 | 65.45 |
| | SEG | 0 | 30.04 |
| | STE | 0 | 1541 |
| | T24 | 0 | 0.28 |
| | TCCSup | 0 | 1.53 |
| | UM-UC3 | 0 | 199.7 |
| Breast | BC173 | 2.26 | 0.1 |
| | BC227 | 1.8 | 0 |
| Colon | CR IGR012P | 8.5 | 0.2 |
| | CR LRB018P | 21.6 | 0 |
| | HCT116 | 0 | 1 |
| | HT29 | 0 | 0 |
| | TC310X6 | 0 | 1 |
| | TC316X5 | 0 | 0.1 |
| | TC320X6 | 4.3 | 14.3 |
| | TC329 | 0 | 2.7 |
| Lung | A549 | 57.8 | 1.9 |
| | CALU3 | 15 | 38.5 |
| | LCF09 | 351.9 | 36.4 |
| | LCF11 | 9 | 66.3 |
| | LCF25 | 0 | 2.01 |
| | NICH460 | 55.2 | 0.35 |
| | SC61 | 0 | 2.63 |
| | SHP77 | 2.4 | 16.2 |
| Placenta | JAR | 37 | 776.9 |
| | JEG-3 | 26.79 | 16.7 |

Pro-EPIL was detected in the supernatant of 16 out of 35 cell lines and hCGβ type II was detected in the supernatant of 32 out of 35 cell lines. Finally, it was striking that, at the exception of the HT 29 colon cancer cell line, all cell lines (34/35) produced either pro-EPIL, hCGβ type II or both. These results are in line with previous data demonstrating that most human cancer cell lines undergo massive genomic hypomethylation. Moreover, results observed with CP antigens pro-EPIL and hCGβ type II can be compared with those observed with the prototypic CT antigen MAGE-1 in various cancer cell lines. While the expression of MAGE-1 is correlated to genome-wide demethylation, it was noteworthy that two melanoma cell lines with low levels of DNA methylation did not express MAGE-1. This observation suggested that global demethylation affects the genome randomly, and therefore does not always lead to the demethylation of the MAGE-1 promoter region. Similarly, our present results show that most cell lines expressed either pro-EPIL or hCGβ type II (34/35 cell lines) but not pro-EPIL and hCGβ type II simultaneously (14/35). As for MAGE-1, these results also suggest that global hypomethylation does not always lead to demethylation of INSL-4, CG3, CG5 or CG8 promoter regions. Thus, for clinical purpose, it is wise to use at least two biomarkers of global hypomethylations (CP antigens) for accurately determining the genome-wide demethylation status of either cancer or placental cells.

### EXAMPLE VI

### Determination of the Expression of hCGβ Type II and Pro-EPIL in Cancer Cell Lines After the Induction of DNA-Demethylation

To further determine the impact of global demethylation on the expression of hCGβ type II and pro-EPIL, we used 5-aza-2′ deoxycytidine (5-aza-dC), a strong inducer of DNA de-methylation and measured the levels of hCGβ type II and pro-EPIL in the supernatants of 10 cancer cell lines of various histological origin, treated at two different doses of 5-aza-dC. On the curve of viability of each cell line treated with 5-aza-dC at increasing doses, the two selected doses that have been selected for studying the effect of global hypomethylation by 5-aza-dC on the production of hCGβ type II and pro-EPIL were the doses corresponding to the 5-aza-dC concentrations at the inflexion point and at the 50% inhibition of cell viability (Inhibition growth Concentration 50% or IC 50), respectively (FIG. 3 and Table 2).

TABLE 2

| | bladder | | breast | colon | | lung | | | placenta | |
|---|---|---|---|---|---|---|---|---|---|---|
| Cell line | HT1376 | OBR | BC173 | CLRB018P | TC320X6 | A549 | LCF09 | SHP77 | JAR | JEG |
| Dose A (μM) | 1.6 | 6.5 | 1.95 | 15.6 | 4 | 12.5 | 5 | 8 | 2 | 2.6 |
| Dose B (μM) | 6.3 | 20 | 117 | 186.2 | 77 | 50 | 25 | 27.5 | 17.7 | 5 |

Briefly, 10,000 cells were plated in 96-well plates in 100 μl medium. After one day, dilution range of 5-aza-dC from 1,000 μM to 0.06 μM were added. After 96 h incubation time, 10 μl of WST-1 were added and optical density (O.D) were measured at 450 nm (FIG. 3 and Table 2).

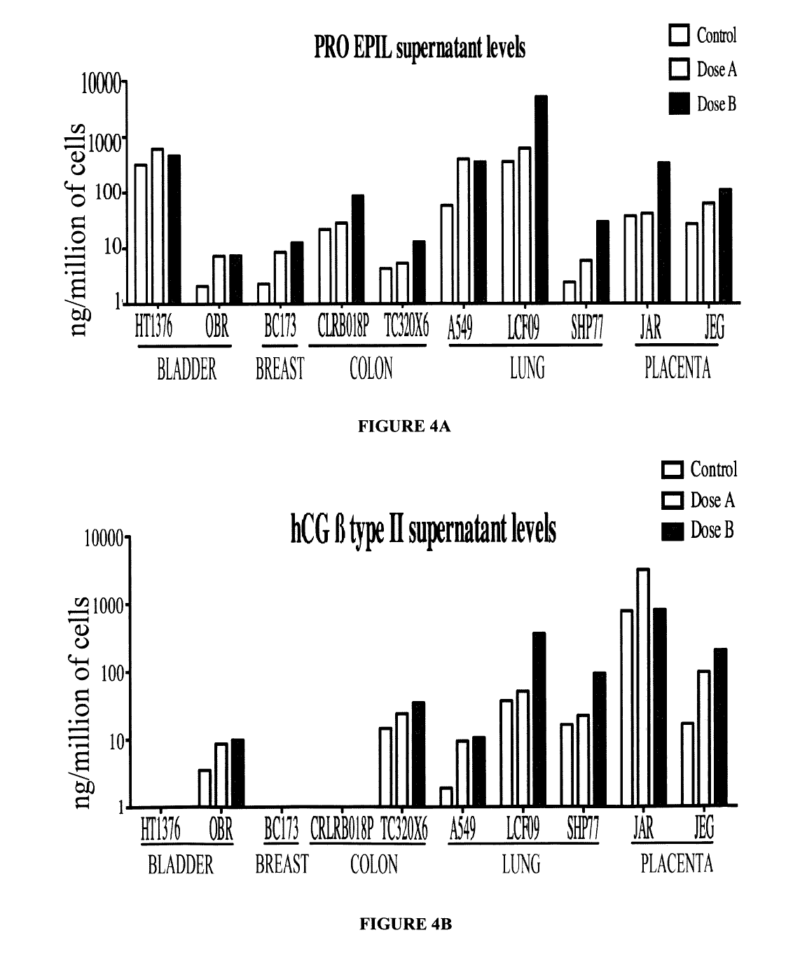

Experiments were performed as previously described [34]. Briefly, in 75 cm² size cell culture flaks, cell lines were grew up to more than 50% confluence before treating them for 72 h with 5-aza-dC at either the doses A corresponding at the Inflection Points, or the doses B corresponding to IC50. Controls were carried with cells cultured in the same experimental conditions but not exposed to 5-aza-dC. After trypsinisation, cells were counted in malassez chamber using trypan blue. Supernatants were concentrated using fast ultracentrifugation based on 10 kDa cutoff. centrifugal filter devices. Centrifugation runs were performed at 3,500 rpm for 30 minutes at 4° C. Then, the pro-EPIL and hCG B type II concentrations were determined by an ELISA immunoassay and an automated KRYPTOR immunoassay, respectively (Table 2, FIG. 4A, FIG. 4B).

FIG. 4A shows that 5-aza-dC induced consistently an increase in the production of pro-EPIL, while it induced an increase in the production of hCGβ type II in 7 out of 10 and cell lines as shown in FIG. 4B

Thus, in addition to cancer diseases, Pro-EPIL and hCGβ type II can also be used as surrogate biomarkers of epigenetic alterations associated with various pathologies of pregnancy including of intrauterine growth retardation, pre-eclampsia or chromosomally abnormal pregnancies. As a first example, it is striking that the transcription levels of both INSL4 and type II CGB genes are increased in chromosomally abnormal pregnancies [34]. Moreover, free hCGβ is already a serum biomarker widely used for the screening of Down's syndrome and it is highly likely that hCGβ type II might constitute a better biomarker of these chromosomally abnormal pregnancies, alone, in combination with pro-EPIL or in combination with other serum markers of epigenetic alterations and global hypomethylation which can be selected by the method of the present invention.

### REFERENCES

1. Nelissen E C, van Montfoort A P, Dumoulin J C, Evers J L. Epigenetics and the placenta. Human reproduction update 2011; 17: 397-417.
2. Reik W. Stability and flexibility of epigenetic gene regulation in mammalian development. Nature 2007; 447: 425-32.
3. Feinberg A P, Tycko B. The history of cancer epigenetics. Nature reviews Cancer 2004; 4: 143-53.
4. Qiu J. Epigenetics: unfinished symphony. Nature 2006; 441: 143-5.
5. Gama-Sosa M A, Midgett R M, Slagel V A, et al. Tissue-specific differences in DNA methylation in various mammals. Biochimica et biophysica acta 1983; 740: 212-9.
6. Ehrlich M. DNA methylation in cancer: too much, but also too little. Oncogene 2002; 21: 5400-13.
7. Ehrlich M, Gama-Sosa M A, Huang L H, et al. Amount and distribution of 5-methylcytosine in human DNA from different types of tissues of cells. Nucleic acids research 1982; 10: 2709-21.
8. Feinberg A P, Vogelstein B. Hypomethylation distinguishes genes of some human cancers from their normal counterparts. Nature 1983; 301: 89-92.
9. Gama-Sosa M A, Slagel V A, Trewyn R W, et al. The 5-methylcytosine content of DNA from human tumors. Nucleic acids research 1983; 11: 6883-94.
10. Ehrlich M, Jiang G, Fiala E, et al. Hypomethylation and hypermethylation of DNA in Wilms tumors. Oncogene 2002; 21: 6694-702.
11. Ferretti C, Bruni L, Dangles-Marie V, Pecking A P, Bellet D. Molecular circuits shared by placental and cancer cells, and their implications in the proliferative, invasive and migratory capacities of trophoblasts. Hum Reprod Update 2007; 13: 121-41.
12. Gama-Sosa M A, Wang R Y, Kuo K C, Gehrke C W, Ehrlich M. The 5-methylcytosine content of highly repeated sequences in human DNA. Nucleic acids research 1983; 11: 3087-95.
13. Nephew K P, Balch C, Skalnik D G. Methyl group acceptance assay for the determination of global DNA methylation levels. Methods in molecular biology 2009; 507: 35-41.
14. Frommer M, McDonald L E, Millar D S, et al. A genomic sequencing protocol that yields a positive display of 5-methylcytosine residues in individual DNA strands. Proc Natl Acad Sci USA 1992; 89: 1827-31.
15. Trinh B N, Long T I, Laird P W. DNA methylation analysis by MethyLight technology. Methods 2001; 25: 456-62.
16. Esteller M, Sanchez-Cespedes M, Rosell R, Sidransky D, Baylin S B, Herman J G. Detection of aberrant promoter hypermethylation of tumor suppressor genes in serum DNA from non-small cell lung cancer patients. Cancer Res 1999; 59: 67-70.
17. Heyn H, Esteller M. DNA methylation profiling in the clinic: applications and challenges. Nat Rev Genet 2012; 13: 679-92.
18. Simpson A J, Caballero O L, Jungbluth A, Chen Y T, Old L J. Cancer/testis antigens, gametogenesis and cancer. Nature reviews Cancer 2005; 5: 615-25.
19. De Smet C, De Backer O, Faraoni I, Lurquin C, Brasseur F, Boon T. The activation of human gene MAGE-1 in tumor cells is correlated with genome-wide demethylation. Proc Natl Acad Sci USA 1996; 93: 7149-53.
20. Issa J P, Gharibyan V, Cortes J, et al. Phase II study of low-dose decitabine in patients with chronic myelogenous leukemia resistant to imatinib mesylate. Journal of clinical oncology 2005; 23: 3948-56.
21. Down T A, Rakyan V K, Turner D J, et al. A Bayesian deconvolution strategy for immunoprecipitation-based DNA methylome analysis. Nature biotechnology 2008; 26: 779-85.
22. Weber M, Davies J J, Wittig D, et al. Chromosome-wide and promoter-specific analyses identify sites of differential DNA methylation in normal and transformed human cells. Nature genetics 2005; 37: 853-62.
23. Stenman U H, Tiitinen A, Alfthan H, Valmu L. The classification, functions and clinical use of different isoforms of HCG. Hum Reprod Update 2006; 12: 769-84.
24. Bellet D H, Ozturk M, Bidart J M, Bohuon C J, Wands J R. Sensitive and specific assay for human chorionic gonadotropin (hCG) based on anti-peptide and anti-hCG monoclonal antibodies: construction and clinical implications. J Clin Endocrinol Metab 1986; 63: 1319-27.
25. Bidart J M, Troalen F, Salesse R, Bousfield G R, Bohuon C J, Bellet D H. Topographic antigenic determinants recognized by monoclonal antibodies on human choriogonadotropin beta-subunit. J Biol Chem 1987; 262: 8551-6.
26. Chassin D, Laurent A, Janneau J L, Berger R, Bellet D. Cloning of a new member of the insulin gene superfamily (INSL4) expressed in human placenta. Genomics 1995; 29: 465-70.
27. Bellet D, Lavaissiere L, Mock P, et al. Identification of pro-EPIL and EPIL peptides translated from insulin-like 4 (INSL4) mRNA in human placenta. J Clin Endocrinol Metab 1997; 82: 3169-72.
28. Bieche I, Laurent A, Laurendeau I, et al. Placenta-specific INSL4 expression is mediated by a human endogenous retrovirus element. Biol Reprod 2003; 68: 1422-9.
29. Brandt B, Roetger A, Bidart J M, et al. Early placenta insulin-like growth factor (pro-EPIL) is overexpressed and secreted by c-erbB-2-positive cells with high invasion potential. Cancer Res 2002; 62: 1020-4.
30. Mock P, Frydman R, Bellet D, et al. Pro-EPIL forms are present in amniotic fluid and maternal serum during normal pregnancy. J Clin Endocrinol Metab 1999; 84: 2253-6.
31. Grigoriu A, Ferreira J C, Choufani S, Baczyk D, Kingdom J, Weksberg R. Cell specific patterns of methylation in the human placenta. Epigenetics 2011; 6: 368-79.
32. Paz M F, Fraga M F, Avila S, et al. A systematic profile of DNA methylation in human cancer cell lines. Cancer Res 2003; 63: 1114-21.
33. Sadikovic B, Haines T R, Butcher D T, Rodenhiser D I. Chemically induced DNA hypomethylation in breast carcinoma cells detected by the amplification of intermethylated sites. Breast Cancer Res 2004; 6: R329-37.
34. Mock P, Frydman R, Bellet D, et al. Expression of pro-EPIL peptides encoded by the insulin-like 4 (INSL4) gene in chromosomally abnormal pregnancies. J Clin Endocrinol Metab 2000; 85: 3941-4.
35. Wapner R, Thom E, Simpson J L, et al. First-trimester screening for trisomies 21 and 18. N Engl J Med 2003; 349: 1405-13.

1.-17. (canceled)

18. A method for the selection of a serum biomarker of epigenetic alterations, particularly of global hypomethylation, said method comprising the steps of:
a) selecting a gene which is expressed as a protein or a peptide in a cancer and/or a placental cell, and whose expression in said cell is associated to the genome hypomethylation of the expression regulation system of said gene and wherein the genome hypomethylation of the expression regulation system of said gene is the hypomethylation of the retrotransposons that regulate said gene; wherein said gene is not expressed in more than two non germline normal tissues;
b) optionally, determining the increase of the protein encoded by said gene selected in step a) in a serum or plasma sample of a patient known to exhibit a cancer or an abnormal pregnancy linked to a pathology of the placenta, preferably compared to a healthy patient serum or plasma sample; and
c) selecting said protein, encoded by said gene as a serum biomarker of genome hypomethylation, whether this gene satisfy the step a) and step b) selection.

19. A method for the selection of a serum biomarker of genome hypomethylation according to claim 18, wherein said retrotransposons that regulate said gene are selected from the group consisting of KCNH5, ERVWEI, EDNRB, PTN, and MIDI retrotransposons.

20. A method for the selection of a serum biomarker of genome hypomethylation according to claim 18 or claim 19, wherein said hypomethylation is measured by a method chosen in the group consisting of: HPLC, Southern blot, methyl group acceptance assay, direct bisulphite sequencing, methylation sensitive PCR, quantitative PCR and the use of monoclonal antibodies against 5-methylcytosine.

21. A method for diagnosing a cancer or abnormal pregnancy linked to a pathology of the placenta associated to the genome hypomethylation of cell in a patient comprising the steps of:
a) selecting at least one serum biomarker of genome hypomethylation, selected by the method of claim 18;
b) determining the genome hypomethylation status of the expression regulation system of the cell in a sample from said patient; and
c) determining the presence or the level of said biomarker selected in step a) in a serum or plasma sample of said patient,
wherein the increase of said biomarker in said patient sample, compared with said biomarker level found for healthy patient serum or plasma sample, is indicative of said cancer or abnormal pregnancy linked to a pathology of the placenta.

22. A method of detecting a predisposition to, or the incidence of, abnormal pregnancy linked to a pathology of the placenta or cancer pathology in a patient which comprises:
a) selecting at least one serum biomarker of genome hypomethylation selected by the method of claim 18; and
b) determining the presence or the level of said biomarker selected in step a) in a serum or plasma sample of said patient,
wherein the significant increase of said biomarker in said patient sample compared with the level of said biomarker found for healthy patient serum or plasma sample, is indicative of a predisposition to, or the incidence of, abnormal pregnancy linked to a pathology of the placenta or cancer.

23. A method for predicting the effect of a demethylating agent, comprising the following steps:
a) selecting at least one serum biomarker of genome hypomethylation, by a method of claim 18; and
b) determining the presence or the level of said biomarker selected in step a) in a serum or plasma sample of said patient having being treated with said agent,
wherein the increase of said biomarker in said patient sample, compared with the level of said biomarker found for in a serum or plasma sample of said patient before said treatment, is indicative of the effect of said agent.

24. A method for following the effect of a demethylating agent, comprising the following steps:
a) selecting at least one serum biomarker of genome hypomethylation selected by the method of claim 18; and
b) determining the presence or the level of said biomarker selected in step a) in a serum or plasma sample of said patient having being treated with said agent,
wherein the increase of said biomarker in said patient sample, compared with the level of said biomarker found for in a serum or plasma sample of said patient before said treatment, is indicative of the predicted effect or of the effect of said agent.

25. The method according to claim 18, wherein said at least one serum biomarker of genome hypomethylation, is selected from the group consisting of the INSL4 (pro-EPIL) and hCGβ type II biomarker.

26. The method according to claim 18, wherein in step b) or c), the determination of the presence or the level of said biomarker in a serum or plasma sample of said patient is carried out by the use of antibody specifically directed against the selected serum biomarker of genome hypomethylation.

27. A method for diagnosing a cancer or abnormal pregnancy linked to a pathology of the placenta associated to genome hypomethylation of cell in a patient, said method comprises the step of:
a) specifically detecting or quantifying the presence of the pro-EPIL peptide and/or of hCGβ type II subunit in a serum or plasma sample from said patient susceptible of containing pro-EPIL and/or hCGβ subunits type II, by a method which implements the use of a polyclonal or a monoclonal antibody (mAb) specifically directed to said pro-EPIL peptide, or to a specific fragment thereof, and/or the use of a polyclonal antibody or mAb specifically directed to said hCGβ type II subunit, or to a specific fragment thereof.

28. A method for detecting a predisposition to, or the incidence of an abnormal pregnancy linked to a pathology of the placenta or cancer pathology in a patient, said method comprises the step of:
a) specifically detecting or quantifying the presence of the pro-EPIL peptide and/or of hCGβ type II subunit peptide, or specific fragment thereof, in a serum or plasma sample from said patient susceptible of containing pro-EPIL and/or hCGβ subunits type II peptide, by a method which implements the use of a polyclonal antibody or a mAb specifically directed to said pro-EPIL peptide, or to a specific fragment thereof, and/or the use of a polyclonal antibody or a mAb specifically directed to said hCGβ type II subunit, or to a specific fragment thereof.

29. The method according to claim 27 or claim 28, wherein said antibody specifically directed to said pro-EPIL peptide or to said hCGβ type II subunit peptide is selected from the group consisting of:
a) polyclonal, monoclonal chimeric or humanized antibodies able to selectively bind, or which selectively bind to an epitope-containing a pro-EPIL polypeptide comprising a contiguous span of at least 9 to 10 amino acids of a pro-EPIL fragment, particularly of a fragment of at least the chain A, B or C of the human pro-EPIL; or
b) polyclonal, monoclonal chimeric or humanized antibodies able to selectively bind hCGβ type II subunit, or a specific fragment thereof, preferably able to selectively bind a discontinuous epitope that comprises region 1-7 with a lysine and a proline residue at position 2 and 4 respectively and region 82-92 of hCGβ, or an anti hCGβ type II-mAb specific binding fragment thereof.

30. The method according to claim 27 or 28, wherein said antibody specifically directed to said pro-EPIL peptide or to said hCG(type II subunit peptide is selected from the group consisting of:
a) polyclonal, monoclonal chimeric or humanized antibodies able to selectively bind, or which selectively bind to an epitope-containing a pro-EPIL polypeptide comprising a contiguous span of at least 9 to 10 amino acids of a pro-EPIL fragment, particularly of a fragment of at least the chain A, B or C of the human pro-EPIL; or
b) polyclonal, monoclonal chimeric or humanized antibodies able to selectively bind hCGβ type II subunit, or a specific fragment thereof, preferably able to selectively bind a discontinuous epitope that comprises region 1-7 with a lysine and a proline residue at position 2 and 4 respectively and region 82-92 of hCGβ, or an anti hCGβ type II-mAb specific binding fragment thereof
wherein this antibody specifically directed to said hCGβ type II subunit peptide is selected from the group consisting of:
the mAb FBT-11-II produced by the hybridoma deposited with the CNCM (Collection Nationale de Cultures de Microorganismes, Institut Pasteur, 25 rue du Docteur Roux, F-75724 PARIS Cedex 15) on Mar. 9, 2010 under the number 1-4281;
the mAb FBT-11 produced by the hybridoma deposited with the CNCM on Oct. 3, 1985 under the number 1-489;
a recombinant mAb having a sequence comprising at least the 6 CDRs (Complementary Determining Region) of the mAb FBT-11-II produced by the hybridoma deposited under the number 1-4281 or at least the 6 CDRs of the the mAb FBT-11 produced by the hybridoma deposited under the number 1-489;
a mAb specifically directed to a discontinuous epitope that comprises region 1-7 with a lysine and a proline residue at position 2 and 4 respectively and region 82-92 of hCGβ, wherein this antibody is produced by an hybridoma obtained from a mouse which has been prior immunized with an antigen comprising at least the fragments 1-7 with a lysine and a proline residue at position 2 and 4 respectively and 82-92 of hCGβ, said hybridoma being selected based on the capability of its secreted mAb to specifically recognizing the fragments 1-7 with a lysine and a proline residue at position 2 and 4 of the hCGβ type II; and
a hCGβ type II-binding fragment thereof.

31. The method of claim 18, claim 27, or claim 28, wherein the determination of the presence or the level of said biomarker in a serum or plasma sample of said patient is carried out by immunoassay, preferably by ELISA immunoassay or radioimmunoassay in blood serum or plasma.

32. A kit for diagnosing a pathology associated to genome hypomethylation of cell in a patient, or for detecting a predisposition to, or the incidence of, abnormal pregnancy linked to a pathology of the placenta or cancer pathology in a patient, said kit comprising:
a) an antibody directed specifically against the pro-EPIL peptide, or specific fragment thereof; and
b) an antibody directed specifically against the hCGβ-type II subunit peptide, or specific fragment thereof.

33. Use of a kit according to claim 32, for diagnosing cancer or abnormal pregnancy linked to a pathology of the placenta associated to genome hypomethylation of cells, for detecting a predisposition to abnormal pregnancy linked to a pathology of the placenta or cancer pathology or for detecting the incidence of abnormal pregnancy linked to a pathology of the placenta or cancer pathology in a patient.
